(19) United States
(12) Patent Application Publication
(10) Pub. No.: US 2024/0010758 A1
(43) Pub. Date: Jan. 11, 2024

(54) SYNERGISTIC ANTIFOULANT COMPOSITIONS AND METHODS OF USING THE SAME

(71) Applicant: Ecolab USA Inc., St. Paul, MN (US)

(72) Inventors: Meng KONG, Shanghai (CN); Kameswara VYAKARANAM, Sugar Land, TX (US)

(73) Assignee: Ecolab USA Inc., St. Paul, MN (US)

(21) Appl. No.: 18/348,074

(22) Filed: Jul. 6, 2023

(30) Foreign Application Priority Data
Jul. 8, 2022 (CN) 202210804720.8

(51) Int. Cl. C08F 2/38; C07D 279/26; C07D 279/22; C07D 279/20

(52) U.S. Cl. CPC C08F 2/38; C07D 279/26; C07D 279/22; C07D 279/20

(57) ABSTRACT

Polymerization inhibitor compositions are provided. The polymerization inhibitor compositions include at least a first inhibitor compound having a phenothiazine or a derivative thereof, a second inhibitor compound having a phenylenediamine or a derivative thereof, and a solvent. Methods of inhibiting the polymerization of monomers using the compositions of the disclosure are also provided. The methods of inhibiting polymerization of monomers include a step of adding a composition of the disclosure to the monomer. In some instances, the monomer is an ethylenically unsaturated monomer. Such ethylenically unsaturated monomers include, but are not limited to, (meth)acrylic acid, methyl methacrylate, acrylic acid, acrylic acid esters, methacrylamide sulfate, vinyl acetate, acrylonitrile, acrolein, acrylates, methacrylates, 1,3-butadiene, styrene, isoprene, and combinations thereof. Methods of preparing the polymerization inhibitors and compositions of the disclosure are also provided.

### FIELD OF THE INVENTION

The present disclosure generally relates to compositions that include a blend of polymerization inhibitors and methods of using the same. More particularly, the present disclosure relates to compositions that include at least one compound having a phenothiazine and phenylenediamine, in a solvent, useful for inhibiting polymerization of ethylenic unsaturated monomers. The present disclosure further relates to methods of preparing compositions having a phenothiazine or derivative thereof and a phenylenediamine or derivative thereof in a functional solvent.

### BACKGROUND

The manufacture of ethylenically unsaturated monomers typically comprises three stages: reaction, recovery, and purification. Distillation operations at elevated temperatures are often involved in the recovery and the purification stages. Ethylenically unsaturated monomers, such as vinyl acetate, acrylate, and methacrylate monomers, can be present in processing streams or in refined products made by various chemical industrial processes. However, these monomer types may undesirably polymerize through radical polymerization especially at elevated temperature and when polymerization initiators are present. As a result, solid deposits of polymer can form on the surface of the process equipment during industrial manufacture, processing, handling, or storage. The resulting polymers can be problematic and lead to equipment “fouling” and product contamination. Accordingly, this can necessitate treating the apparatus to remove the polymer, or may necessitate processing steps to remove the polymer from compositions streams or stored compositions. These undesirable polymerization reactions result in a loss in production efficiency because they consume valuable reagents and additional steps may be required to clean equipment and/or to remove the undesired polymers.

The premature polymerization of these monomers is generally controlled by dosing polymerization inhibitors capable of reducing the premature polymerization of the monomers. Conventional polymerization inhibitors include stable free radicals that can effectively scavenge carbon-centered radicals. Conventional polymerization inhibitors, such as 4-hydroxy-2,2,6,6-tetramethylpiperidin-1-oxyl (HTEMPO) and 4-oxo-2,2,6,6-tetramethylpiperidin-1-oxyl (OTEMPO), generally degrade and lose their efficacy as polymerization inhibitors under acidic environments. Thus, there is a need to develop new polymerization inhibitors, particularly inhibitors that are stable under acidic conditions.

Methyl methacrylate plants are troubled by fouling issues which can occur in methacrylic acid (MAA) production, esterification units, purification units and spent acid units. Phenothiazine (PTZ) solution is typically prepared at a low concentration because of poor solubility, however, a concentrated formula is needed to improve antifouling applications.

### BRIEF SUMMARY

Compositions for inhibiting the polymerization of monomers are disclosed herein. The compositions include a first inhibitor compound having phenothiazine or a derivative thereof, a second inhibitor compound having phenylenediamine or a derivative thereof, and a solvent selected from the group consisting of a pyrrolidone, an ester that is a phthalate, a C₃-C₁₀, preferably, a C₅-C₁₀, ether such as butyl carbitol, or a combination thereof. In some embodiments, the compositions are useful for inhibiting the polymerization of ethylenically unsaturated monomers including (meth)acrylic acid, methyl methacrylate, acrylic acid, acrylic acid esters, methacrylamide sulfate, vinyl acetate, acrylonitrile, acrolein, acrylates, methacrylates, 1,3-butadiene, styrene, isoprene, and combinations thereof.

In some aspects, the first inhibitor compound is of formula (I):

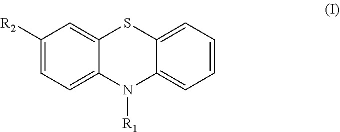

or a derivative thereof, wherein R₁ is selected from the group consisting of H and a C₁-C₈ alkyl group, and wherein R₂ is selected from the group consisting of H, C₁-C₂₀ alkyl, hydroxyl, amino, amido, ester and carboxylate. In certain aspects, the R₂ substituents, such as the alkyl group, include from about 1 to about 5 carbon atoms and may be linear or branched. It should be further understood that, in some embodiments, derivatives of the second inhibitor compound can include where the R₁ groups can be linked to the nitrogen via an additional C₁-C₈ alkyl, straight or branched, linker and/or can be further substituted by C₁₋₂₀ alkyl or substituted or unsubstituted heterocyclyl.

In some aspects, the second inhibitor compound is a phenylenediamine of formula (II):

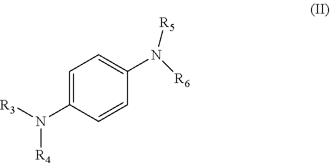

wherein each of R₃, R₄, R₅ and R₆ is independently selected from the group consisting of hydrogen, C₁-C₁₀ alkyl, C₁-C₁₀ aryl, C₁-C₁₀ heteroaryl, hydroxyl, C₁-C₁₀ alkoxyl, ester, and carboxylate. Preferably, the carbon ranges are C₁-C₅.

In some aspects, the solvent is selected from the group consisting a pyrrolidone, an ester that is a phthalate, a C₃-C₁₀, preferably, a C₅-C₁₀, ether such as butyl carbitol, and combinations thereof.

In some embodiments, the compositions of the disclosure demonstrate synergy with respect to their ability to inhibit polymerization of a monomer. For example, in some embodiments, the compositions of the disclosure demonstrate greater polymerization inhibition than the individual components present within the composition, controlling for the dosage of the active components.

In some embodiments, the compositions of the disclosure are active even under acidic conditions, unlike conventional polymerization inhibitors known in the art. Thus, in some embodiments, the compositions of the disclosure also include one or more acids.

Methods of inhibiting the polymerization of a monomer are also disclosed herein. The methods of inhibiting the polymerization of a monomer include the step of adding a composition of the disclosure to the monomer. Addition of the polymerization inhibitor composition of the disclosure to the monomer inhibits polymerization of the monomer.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### DETAILED DESCRIPTION

Various embodiments of the present disclosure are described below. The relationship and functioning of the various elements of the embodiments may better be understood by reference to the following detailed description. However, embodiments are not limited to those explicitly described herein.

The present disclosure relates to compositions that include a blend of polymerization inhibitors and methods of using the same to inhibit the polymerization of ethylenically unsaturated monomers. Polymerization inhibitor compositions of the present disclosure include at least one compound having a phenothiazine or a derivative thereof and at least one compound having a phenylenediamine or a derivative thereof in a solvent. The polymerization inhibitor compositions can be blends of multiple components, including components in addition to the aforementioned compounds having a phenothiazine or derivative thereof and a phenylenediamine or derivative thereof.

A “polymerization inhibitor,” in the presence of polymerizable monomers, inhibits the formation of a polymer from those monomers during the induction time. After the induction time has lapsed, the polymer's formation occurs at substantially the same rate that it would form at in the absence of the polymerization inhibitor.

Polymerization inhibitors and polymerization retarders can be considered generally as “antipolymerants” which are compounds that can inhibit or reduce the formation of polymers from one or more radically polymerizable compounds.

The term “fouling” refers to the formation of polymers, prepolymers, oligomer and/or other materials, which would become insoluble in and/or precipitate from a stream and deposit on equipment under the conditions of operation of the equipment. In turn, the inhibitor, retarder, and amine stabilizer components and compositions of the disclosure can be referred to as “antifouling” as they inhibit or reduce such formation.

### Compositions of the Disclosure

The present disclosure relates to compositions for inhibiting monomer polymerization where the compositions include a first inhibitor compound having a phenothiazine or a derivative thereof and a second inhibitor compound having a phenylenediamine or a derivative thereof in a solvent. In some embodiments, the compositions are for inhibiting monomer polymerization, where the monomer is an ethylenically unsaturated monomer. For example, the compositions of the disclosure are useful for inhibiting polymerization of ethylenically unsaturated monomers including, but not limited to, (meth)acrylic acid, methyl methacrylate, acrylic acid, acrylic acid esters, methacrylamide sulfate, vinyl acetate, acrylonitrile, acrolein, acrylates, methacrylates, 1,3-butadiene, styrene, isoprene, and combinations thereof.

In some embodiments, the first inhibitor compound having a phenothiazine is a compound of formula (I):

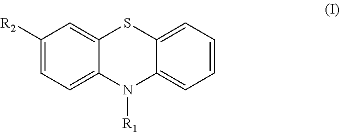

wherein R₁ is selected from the group consisting of H and a C₁-C₈ alkyl group, and wherein R₂ is selected from the group consisting of H, C₁-C₂₀ alkyl, hydroxyl, amino, amido, ester and carboxylate. In certain aspects, the R₂ substituents, such as the alkyl group, include from about 1 to about 5 carbon atoms and may be linear or branched. It should be further understood that, in some embodiments, derivatives of the second inhibitor compound can include where the R₁ groups can be linked to the nitrogen via an additional C₁-C₈ alkyl, straight or branched, linker and/or can be further substituted by C₁-C₂₀ alkyl or substituted or unsubstituted heterocyclyl.

In some embodiments, the second inhibitor compound having a phenylenediamine or a derivative thereof of is a compound of formula (II):

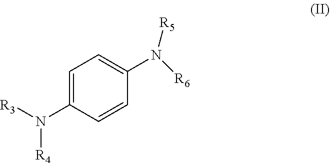

wherein each of R₃, R₄, R₅ and R₆ is independently selected from the group consisting of hydrogen, C₁-C₁₀ or C₁-C₅ alkyl, C₁-C₁₀ or C₁-C₅ aryl, C₁-C₁₀ or C₁-C₅ heteroaryl, hydroxyl, C₁-C₁₀ or C₁-C₅ alkoxyl, an ester, and a carboxylate.

In some embodiments, the first inhibitor compound having a phenothiazine or derivative thereof is a compound selected from the group consisting of:

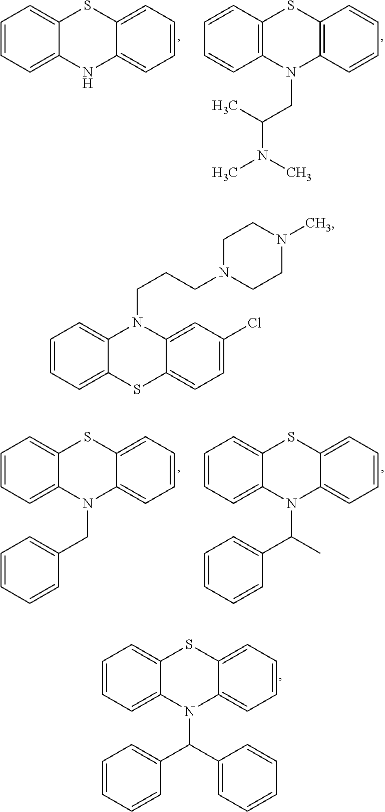

and combinations thereof.

In some embodiments, the second inhibitor compound having a phenylenediamine or derivative thereof is a compound selected from the group consisting of: N,N-di-s-butyl-p-phenylenediamine, N,N′-Di-sec-butyl-p-phenylenediamine, N-(1,4-Dimethylpentyl)-N′-Phenyl-1,4-Phenylenediamine, and combinations thereof.

In certain embodiments, other inhibitors, such as antioxidants, could be added and/or present in the compositions disclosed herein. Antipolymerants, such as HTEMPO, could also be added and in some embodiments, they are added as a separate stream. Dispersants, such as tall oil fatty acid or oleic acid imidazolines, could also be added and/or present in the compositions disclosed herein. In certain embodiments, the composition can further include an antioxidant. In certain aspects, all of the antifouling compounds can be blended and added in a single stream.

In some embodiments, the solvent can include a pyrrolidone, an ester that is a phthalate, a C₃-C₁₀, preferably, a C₅-C₁₀, ether such as butyl carbitol, or a combination thereof. In certain embodiments, the pyrrolidone comprises N-methylpyrrolidine (CAS No. 120-94-5).

In some embodiments, the pyrrolidone of formula (III):

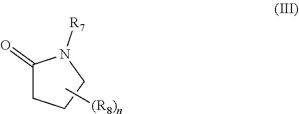

wherein R₇ is selected from the group consisting of hydrogen, C₁-C₈ alkyl, aryl, and heteroaryl, wherein R₈ is selected from the group consisting of hydrogen, C₁-C₈ alkyl, aryl, and heteroaryl, and wherein n is 0, 1, 2, 3, or 4.

In some embodiments, the pyrrolidone is of formula (IIIa):

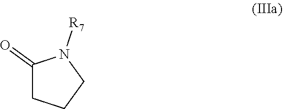

In some embodiments, the pyrrolidone is selected from the group consisting of N-methyl-2-pyrrolidone, 3-hydroxy-2-pyrrolidinone, 1-butylpyrrolidin-2-one, and combinations thereof.

In some embodiments, the phthalate is a C₁-C₂₀ phthalate such as, by way of example, but not limitation, dimethyl phthalate or dibutyl phthalate.

In some embodiments, the composition unexpectedly demonstrates synergy, where the combination of the first and second inhibitor compounds produces a greater degree of polymerization inhibition than would be expected for the combination.

In some embodiments, the first inhibitor compound having a phenothiazine or a derivative thereof is present in the composition at a concentration of about 0.01% by weight to about 60% by weight. In some embodiments, the first inhibitor compound having a phenothiazine or a derivative thereof is present in the composition at a concentration of about 0.01% by weight to about 50% by weight. In some embodiments, the first inhibitor compound having a phenothiazine or derivative thereof is present in the composition at a concentration of about by weight to about 40% by weight. In some embodiments, the first inhibitor compound having a phenothiazine or derivative thereof is present in the composition at a concentration of about 0.01% by weight to about 30% by weight. In some embodiments, the first inhibitor compound having a phenothiazine or derivative thereof is present in the composition at a concentration of about 0.01% by weight to about 20% by weight. In some embodiments, the first inhibitor compound having a phenothiazine or derivative thereof is present in the composition at a concentration of about 0.01% by weight to about 10% by weight. In some embodiments, the first inhibitor compound having a phenothiazine or a derivative thereof is present at a concentration of about 5% to about 40% by weight.

For example, in certain embodiments, the first inhibitor compound having a phenothiazine or a derivative thereof is present in the composition at a concentration of about by weight, about 0.1% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, or about 60% by weight. In certain embodiments, the first inhibitor compound having a phenothiazine or a derivative thereof includes at least 2% PTZ, at least 3% PTZ, at least 5% PTZ, at least 10% PTZ or more up to 10%, 20%, 30%, 40%, 50%, or 60% PTZ by weight.

In some embodiments, the second inhibitor compound having a phenylenediamine or a derivative thereof is present in the composition at a concentration of about 0.01% by weight to about 20% by weight. In some embodiments, the second inhibitor compound having a phenylenediamine or a derivative thereof is present in the composition at a concentration of about by weight to about 10% by weight.

For example, in certain embodiments, the second inhibitor compound having a phenylenediamine or a derivative thereof is present in the composition at a concentration of about by weight, about 0.1% by weight, about 1% by weight, about 5% by weight, about 10% by weight, about 15% by weight, or about 20% by weight.

In some embodiments, a mole ratio of the first inhibitor compound having a phenothiazine or a derivative thereof to the second inhibitor compound having a phenylenediamine or a derivative thereof is about 100:0.01 to about 0.01:100. In some embodiments, a mole ratio of the first inhibitor compound having a phenothiazine or a derivative thereof to the second inhibitor compound having a phenylenediamine or a derivative thereof is about 100:1 to about 1:100. In some embodiments, a mole ratio of the first inhibitor compound having a phenothiazine or a derivative thereof to the second inhibitor compound having a phenylenediamine or a derivative thereof is about 90:1 to about 1:90. In some embodiments, a mole ratio of the first inhibitor compound having a phenothiazine or a derivative thereof to the second inhibitor compound having a phenylenediamine or a derivative thereof is about 80:1 to about 1:80. In some embodiments, a mole ratio of the first inhibitor compound having a phenothiazine or a derivative thereof to the second inhibitor compound having a phenylenediamine or a derivative thereof is about 70:1 to about 1:70. In some embodiments, a mole ratio of the first inhibitor compound having a phenothiazine or a derivative thereof to the second inhibitor compound having a phenylenediamine or a derivative thereof is about 60:1 to about 1:60. In some embodiments, a mole ratio of the first inhibitor compound having a phenothiazine or a derivative thereof to the second inhibitor compound having a phenylenediamine or a derivative thereof is about 50:1 to about 1:50. In some embodiments, a mole ratio of the first inhibitor compound having a phenothiazine or a derivative thereof to the second inhibitor compound having a phenylenediamine or a derivative thereof is about 40:1 to about 1:40. In some embodiments, a mole ratio of the first inhibitor compound having a phenothiazine or a derivative thereof to the second inhibitor compound having a phenylenediamine or a derivative thereof is about 30:1 to about 1:30. In some embodiments, a mole ratio of the first inhibitor compound having a phenothiazine or a derivative thereof to the second inhibitor compound having a phenylenediamine or a derivative thereof is about 20:1 to about 1:20. In some embodiments, a mole ratio of the first inhibitor compound having a phenothiazine or a derivative thereof to the second inhibitor compound having a phenylenediamine or a derivative thereof is about 10:1 to about 1:10. In some embodiments, a mole ratio of the first inhibitor compound having a phenothiazine or a derivative thereof to the second inhibitor compound having a phenylenediamine or a derivative thereof is about 5:1 to about 1:5. In some embodiments, a mole ratio of the first inhibitor compound having a phenothiazine or a derivative thereof to the second inhibitor compound having a phenylenediamine or a derivative thereof is about 1:1.

In certain embodiments, a mole ratio of the first inhibitor compound having a phenothiazine or a derivative thereof to the second inhibitor compound having a phenylenediamine or a derivative thereof is about 0.5:1 to about 5:1.

In certain embodiments, a mole ratio of the first inhibitor compound having a phenothiazine or a derivative thereof to the second inhibitor compound having a phenylenediamine or a derivative thereof is about 2:1 or about 8.85:1. By way of example, but not limitation, the mole ratio of the first inhibitor compound having a phenothiazine or a derivative thereof to the second inhibitor compound having a phenylenediamine or a derivative thereof can be about 2:1 or about 8:1

In some embodiments, a weight ratio of the first inhibitor compound having a phenothiazine or a derivative thereof to the second inhibitor compound having a phenylenediamine or a derivative thereof is about 8:1.

In some embodiments, the solvent is between 1% and 99% of the composition by weight. In some embodiments, the solvent is between 1% and 90% of the composition by weight. In some embodiments, the solvent is between 1% and 80% of the composition by weight. In some embodiments, the solvent is between 1% and 70% of the composition by weight. In some embodiments, the solvent is between 1% and 60% of the composition by weight. In some embodiments, the solvent is between 1% and 50% of the composition by weight. In some embodiments, the solvent is between 1% and 40% of the composition by weight. In some embodiments, the solvent is between 1% and 30% of the composition by weight. In some embodiments, the solvent is between 1% and 20% of the composition by weight. In some embodiments, the solvent is between 1% and 10% of the composition by weight. In some embodiments, the solvent is between 1% and 5% of the composition by weight.

For example, in certain embodiments, the solvent is present in the composition at a concentration of about about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 70% by weight, about 80% by weight, about 90% by weight, or about 99% by weight.

The composition may optionally also include one or more additional solvents. In certain embodiments, the additional solvent can be selected from water, an ester, an aromatic, an aliphatic, an amide, an acylamide, an alcohol, a liquid polymer, and combinations thereof. Preferably, where an alcohol is used, the alcohol is the same as is used in the manufacturing process such as, by way of example, but not limitation, in the (meth)acrylate manufacturing process.

In some embodiments, the composition also includes one or more ethylenically unsaturated monomers. One of ordinary skill in the art will appreciate that there are many ethylenically unsaturated monomers that are compatible with the compositions of the disclosure. For example, in some embodiments, the one or more ethylenically unsaturated monomers are selected from methyl methacrylate, acrylic acid, acrylic acid esters, methacrylamide sulfate, acrolein, acrylates, methacrylates, vinyl acetate, acrylonitrile, acrylates, methacrylates, 1,3-butadiene, styrene, isoprene, (meth)acrylic acid, and combinations thereof. In certain embodiments, the composition also includes vinyl acetate. In certain embodiments, the composition also includes acrylonitrile. In certain embodiments, the composition also includes acrylates. In certain embodiments, the composition also includes methacrylates. In certain embodiments, the composition also includes 1,3-butadiene. In certain embodiments, the composition also includes styrene. In certain embodiments, the composition also includes isoprene. In certain embodiments, the composition also includes (meth)acrylic acid.

In some embodiments, the composition does not include a phenol, a nitroxyl radical, a nitroso group compound, or hydroquinone.

The compositions of the disclosure are stable and remain useful polymerization inhibitors even under acidic conditions. Thus, the compositions of the disclosure are useful for inhibiting the premature polymerization of monomers during manufacturing process, particularly those that are performed under acidic conditions. For example, the compositions of the disclosure are useful for preventing polymerization of acrylates, which may include, but are not limited to, acrylonitrile, acrylic acid, methyl methacrylic acid and its esters, and vinyl acetate.

In certain embodiments, the compositions of the disclosure are generally stable under acidic conditions, a significant improvement over conventional polymerization inhibitors known in the art. Thus, in some embodiments, the composition also includes one or more acids. For example, in some embodiments, the composition also includes one or more acids selected from the group consisting of mineral acids and carboxylic acids. Mineral acids include, but are not limited to, hydrochloric acid, hydrofluoric acid, hydrobromic acid, hydroiodic acid, nitric acid, phosphoric acid, sulfuric acid, boric acid, perchloric acid, and the like. Carboxylic acids include, but are not limited to, formic acid, acetic acid, propionic acid, butyric acid, valeric acid, caproic acid, ethanic acid, caprylic acid, undecylic acid, lauric acid, oxalic acid, malonic acid, succinic acid, glutaric acid, adipic acid, pimelic acid, suberic acid, and the like. In some embodiments, the compositions of the disclosure also include hydrochloric acid. In some embodiments, the compositions of the disclosure also include nitric acid. In some embodiments, the compositions of the disclosure also include phosphoric acid. In some embodiments, the compositions of the disclosure also include sulfuric acid. In some embodiments, the compositions of the disclosure also include acetic acid. In some embodiments, the compositions of the disclosure also include propionic acid. In some embodiments, the compositions of the disclosure also include butyric acid. In some embodiments, the compositions of the disclosure also include valeric acid.

In some embodiments, the composition can be combined with or used with additional antifouling agents such as hydroquinone, transition metal salts, an antioxidizer or a defoamer which can be injected together or separately in methods of the disclosure.

### Methods of Using the Compositions of the Disclosure

The present disclosure also relates to methods of inhibiting polymerization of monomers that include adding a composition of the disclosure to the monomer. In some aspects, an effective amount of the composition of the disclosure is added to the monomer, where an effective amount is any amount sufficient to inhibit the polymerization of the monomer.

In some aspects, the monomer is an ethylenically unsaturated monomer. In some aspects the monomer is an ethylenically unsaturated monomer selected from vinyl acetate, acrylonitrile, acrylates, methacrylates, 1,3-butadiene, styrene, isoprene, (meth)acrylic acid, and combinations thereof are disclosed. In some aspects, the methods disclosed herein are useful in inhibiting the polymerization of vinyl acetate. In some aspects, the methods disclosed herein are useful in inhibiting the polymerization of acrylonitrile. In some aspects, the methods disclosed herein are useful in inhibiting the polymerization of acrylates. In some aspects, the methods disclosed herein are useful in inhibiting the polymerization of methacrylates. In some aspects, the methods disclosed herein are useful in inhibiting the polymerization of 1,3-butadiene. In some aspects, the methods disclosed herein are useful in inhibiting the polymerization of styrene. In some aspects, the methods disclosed herein are useful in inhibiting the polymerization of isoprene. In some aspects, the methods disclosed herein are useful in inhibiting the polymerization of (meth)acrylic acid.

The composition of the disclosure can be added manually or automatically to the fluid. The composition can also be added continuously and/or intermittently. Automatic addition may be accomplished through the use of chemical injection pumps. The chemical injection pumps may be programmed to add particular amounts of the polymerization inhibitor composition, or any components thereof, at certain time intervals to the fluid. In alternate aspects, the chemical injection pumps can be manually controlled to add particular amounts of the polymerization inhibitor composition, or any components thereof, to the fluid. Addition of the presently disclosed polymerization inhibitor compositions to the monomer will thereby inhibit polymerization of the monomer.

In some aspects, the monomer is provided as a neat liquid. In other aspects, the monomer is provided within a solution, hereafter referred to as “the monomer solution”.

In some aspects, the monomer solution also includes one or more additional components selected from an acid, an organic solvent, water, and combinations thereof. For example, in some aspects, the monomer solution includes one or more organic solvents selected from vinyl acetate, dimethyl phthalate, dimethylformamide, toluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In some aspects, the monomer solution includes one or more acids selected from hydrochloric acid, hydrofluoric acid, hydrobromic acid, hydroiodic acid, nitric acid, phosphoric acid, sulfuric acid, boric acid, perchloric acid, formic acid, acetic acid, propionic acid, butyric acid, valeric acid, caproic acid, ethanic acid, caprylic acid, undecylic acid, lauric acid, oxalic acid, malonic acid, succinic acid, glutaric acid, adipic acid, pimelic acid, and suberic acid. In some aspects, the monomer solution includes water.

In some aspects, the monomer solution has a pH value of about 1 to about 7. In some aspects, the monomer solution has a pH value of about 1 to about 6. In some aspects, the monomer solution has a pH value of about 2 to about 6. In some aspects, the monomer solution has a pH value of about 3 to about 6. In some aspects, the monomer solution has a pH value of about 4 to about 6. In some aspects, the monomer solution has a pH value of about 5 to about 6.

In some aspects, the composition is added to the monomer such that the total amount of the first inhibitor compound and the second inhibitor compound are present at about 0.1 ppm to 10,000 ppm. In some aspects, the composition is added to the monomer such that the total amount of the first inhibitor compound and the second inhibitor compound are present at about 0.1 ppm to 5,000 ppm. In some aspects, the composition is added to the monomer such that the total amount of the first inhibitor compound and the second inhibitor compound are present at about 0.1 ppm to 1,000 ppm. In some aspects, the composition is added to the monomer such that the total amount of the first inhibitor compound and the second inhibitor compound are present at about 0.1 ppm to 500 ppm. In some aspects, the composition is added to the monomer such that the total amount of the first inhibitor compound and the second inhibitor compound are present at about 10 ppm to 2,000 ppm.

The methods of the disclosure are useful for inhibiting the premature polymerization of monomers during manufacturing process, particularly those that are performed under acidic conditions. For example, the methods of the disclosure are useful for preventing polymerization of acrylates, which may include, but are not limited to, acrylonitrile, acrylic acid, methyl methacrylic acid and its esters, and vinyl acetate.

The methods of the disclosure are also useful for preventing the premature polymerization of styrene during manufacturing and purification processes.

The methods of the disclosure are also useful in butadiene extraction processes. This utility stems from the balanced partition coefficients between polar organic phases and organic phases.

In some embodiments, a composition of the present disclosure can include the first inhibitor compound in any solvent disclosed or contemplated herein. In other embodiments, a composition of the present disclosure can include the second inhibitor compound in a solvent disclosed or contemplated herein. It should be further understood that, in some embodiments, the separate first and second inhibitor compositions can be injected separately in methods of the present disclosure at different process points, injected together or combined to produce a composition having both the first inhibitor compound and the second inhibitor compound in a solvent of the present disclosure.

In some embodiments, a composition of the present disclosure can include the second inhibitor compound selected from the group consisting of:

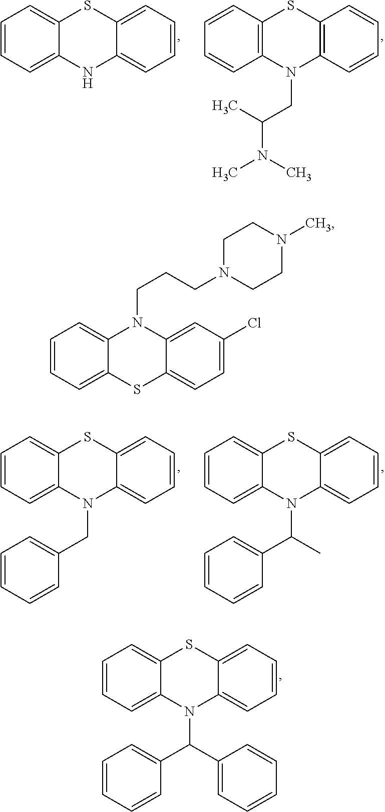

and combinations thereof,
in a solvent selected from the group consisting of a pyrrolidone, an ester that is a phthalate, a C₃-C₁₀, preferably, a C₅-C₁₀, ether such as butyl carbitol, and combinations thereof. In some embodiments, a composition of the present disclosure can include a second inhibitor compound that is

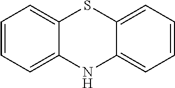 [SMILES: c1ccc2c(c1)Nc1ccccc1S2]

and a solvent that can include N-methyl-2-pyrrolidone. In some embodiments, a composition of the present disclosure can include a second inhibitor compound that is

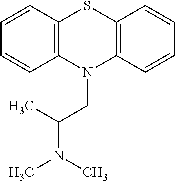 [SMILES: CC(CN1c2ccccc2Sc2ccccc21)N(C)C]

and a solvent that can include N-methyl-2-pyrrolidone. In some embodiments, a composition of the present disclosure can include a second inhibitor compound that is

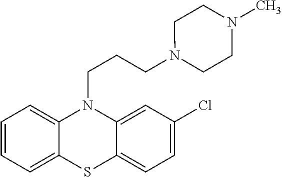 [SMILES: CN1CCN(CCCN2c3ccccc3Sc3ccc(Cl)cc32)CC1]

and a solvent that can include N-methyl-2-pyrrolidone. In some embodiments, a composition of the present disclosure can include a second inhibitor compound that is

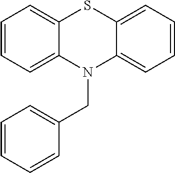 [SMILES: c1ccc(CN2c3ccccc3Sc3ccccc32)cc1]

and a solvent that can include N-methyl-2-pyrrolidone. In some embodiments, a composition of the present disclosure can include a second inhibitor compound that is

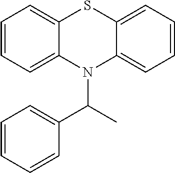 [SMILES: CC(c1ccccc1)N1c2ccccc2Sc2ccccc21]

and a solvent that can include N-methyl-2-pyrrolidone. In some embodiments, a composition of the present disclosure can include a second inhibitor compound that is

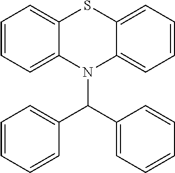 [SMILES: c1ccc(C(c2ccccc2)N2c3ccccc3Sc3ccccc32)cc1]

and a solvent that can include N-methyl-2-pyrrolidone. In certain such embodiments, the first inhibitor compound can be present at about 0.01% to about 60% by weight, preferably about 40% by weight or 65% by weight, of the composition and the functional solvent can be present at about 40% to about 99.99% by weight of the composition. By way of example, but not limitation, the first inhibitor compound can be present at about 65% by weight of the composition when the solvent is a phthalate.

In some embodiments, a composition of the present disclosure can include the second inhibitor compound selected from the group consisting of N,N-di-s-butyl-p-phenylenediamine, N,N′-Di-sec-butyl-p-phenylenediamine, N-(1,4-Dimethylpentyl)-N′-Phenyl-1,4-Phenylenediamine, and combinations thereof, in a solvent selected from the group consisting of a pyrrolidone, an ester that is a phthalate, a C₃-C₁₀, preferably, a C₅-C₁₀, ether such as butyl carbitol, and combinations thereof. In some embodiments, a composition of the present disclosure can include a second inhibitor compound selected from the group consisting of selected from the group consisting of N,N-di-s-butyl-p-phenylenediamine, N,N′-Di-sec-butyl-p-phenylenediamine, N-(1,4-Dimethylpentyl)-N′-Phenyl-1,4-Phenylenediamine, and combinations thereof, and a solvent that can include N-methyl-2-pyrrolidone. In certain such embodiments, the first inhibitor compound can be present at about 0.01% to about 25% by weight of the composition and the functional solvent can be present at about 80% to about 99.99% by weight of the composition.

### Processes for Preparing Compositions of the Disclosure

Compositions of the disclosure can be prepared by simple blending such as, by way of example but not limitation, by dissolving the first inhibitor compound and/or the second inhibitor compound in the same or different solvents and, if in different solvents, combining the solutions. Alternatively, the compositions can be prepared in situ by adding the first inhibitor compound, second inhibitor compound and functional solvent to the process equipment.

Relevant manufacturing process can include, by way of example but not limitation, methods to produce methyl methacrylate, such as the ACH process of the oxidation process which can divided into 4 units—the MAA production unit, esterification unit, purification unit, and spent acid unit. The compositions of the disclosure or components thereof can be injected separately or together. In the purification unit, the first inhibitor compound or the second inhibitor compound can be injected alone or together.

In the ACH manufacturing process, there are 3 reactive monomers-methacrylic acid (MAA), methacrylamide sulfate and methyl methacylate (MMA). All of these monomers are easily polymerized to generate fouling issues and usually the polymerization occurs at the same location, resulting in a complex and variable fouling issue. The fouling issue often occurs in the esterification unit, purification unit, and spent acid unit. All of these units need inhibitors to reduce the fouling issue, such as hydroquinone (HQ), phenothiazine (PTZ), and antioxidizers which can be used. Due to the reactivity difference between mnomers, said inhibitors cannot efficiently reduce or mitigate the fouling issue. For example, HQ and antioxidizers are not efficient at high temperatures for MAA and MMA fouling, PTZ only works for MAA antifouling at high temperature. The compositions of the disclosure and methods disclosed can solve the fouling issues caused by MMA and MAA at the same time. The inhibitor compounds can reduce fouling while the functional solvent can transfer the formed polymer out of the equipment which can maximally reduce fouling to reduce fouling aggregation and improve operation time.

### EXAMPLES

### Example 1—Functional Solvent Performance Test

Dimethyl phthalate (DMP) was tested to assess the solubility of methyl methacrylate (MMA) deposits from the field as described by U.S. Pat. No. 9,884,951 which is incorporated herein by reference in its entirety. Briefly, the solvent was tested by placing a sample of MMA foulant particles in the DMP for 30 minutes to a few hours with observation both at the start of the experiment and after the time period. Dispersion of the foulant without precipitate demonstrated effectiveness of the solvent to remove foulants by gradually dissolving the formed foulant to mitigate blockage issues.

### Example 2—Antifouling Performance Test

An antifouling performance test was performed by a test tube method. Briefly, 30 mL of purified monomer (methacrylic acid) was added into the tube with trace benzoyl peroxide (BPO) (400 ppm), the antifouling formula was then added and nitrogen was bubbled into the liquid to remove oxygen from the liquid, followed by sealing the tube tightly and heating to 125° C. for 3 hours.

70 ppm total (equal parts each—35 ppm—PTZ/PDA if mixed) PTZ, HQ, PDA and PTZ/PDA (phenothiazine/N—N-di-s-butyl-p-phenylenediamine) (left to right) were.

Once polymerization occurs, the liquid is turbid and the formed polymer will aggregate and precipitate to the bottom. The PTZ/PDA combination showed the best performance without visible polymer in the stream, followed by PTZ, while HQ and PDA (N,N-di-s-butyl-p-phenylenediamine) alone is not efficient.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a compound” is intended to include “at least one compound” or “one or more compounds.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A composition for inhibiting monomer polymerization, comprising:
a first inhibitor compound comprising phenothiazine or a derivative thereof;
a second inhibitor compound comprising a phenylenediame or a derivative thereof; and
a solvent comprising a fouling removing agent selected from the group consisting of a pyrrolidone, an ester that is a phthalate, a C₃-C₁₀, preferably, a C₅-C₁₀, ether such as butyl carbitol and combinations thereof.

2. The composition of claim 1, wherein the first inhibitor compound or derivative thereof is of formula (I): 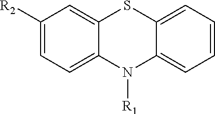
wherein R₁ is selected from the group consisting of H and a C₁-C₈ alkyl group, and wherein R₂ is selected from the group consisting of H, C₁-C₂₀ alkyl, hydroxyl, amino, amido, ester and carboxylate.

3. The composition of claim 1, wherein the first inhibitor compound or derivative thereof is selected from the group consisting of 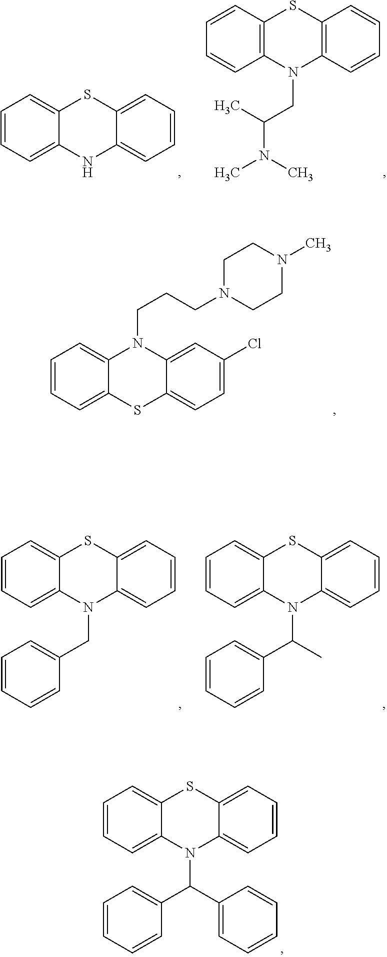
and combinations thereof.

4. The composition of claim 1, wherein the second inhibitor compound or derivative thereof is of formula (II): 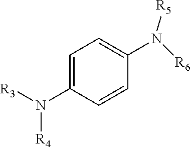
wherein each of R₃, R₄, R₅ and R₆ is independently selected from the group consisting of hydrogen, C₁-C₁₀ alkyl, C₁-C₁₀ aryl, C₁-C₁₀ heteroaryl, hydroxyl, C₁-C₁₀ alkoxyl, ester, and carboxylate.

5. The composition of claim 1, wherein the second inhibitor compound is selected from the group consisting of N,N-di-s-butyl-p-phenylenediamine, N,N′-Di-sec-butyl-p-phenylenediamine, and N-(1,4-DimethylpentyI)-N′-Phenyl-1,4-Phenylenediamine.

6. The composition of claim 1, wherein the solvent comprises pyrrolidone of Formula (III): 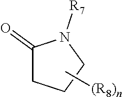
wherein R₇ is selected from the group consisting of hydrogen, C₁-C₈ alkyl, aryl, and heteroaryl, wherein R₈ is selected from the group consisting of hydrogen, C₁-C₈ alkyl, aryl, and heteroaryl, and wherein n is 0, 1, 2, 3, or 4.

7. The composition of claim 1, wherein the solvent comprises pyrrolidone of formula (IIIa): 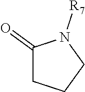
wherein R₇ is selected from the group consisting of hydrogen, C₁-C₈ alkyl, aryl, and heteroaryl.

8. The composition of claim 6, wherein the pyrrolidone is selected from the group consisting of N-methyl-2-pyrrolidone, 3-hydroxy-2-pyrrolidinone, 1-butylpyrrolidin-2-one, and combinations thereof.

9. The composition of claim 1, wherein the solvent comprises a phthalate, and wherein the phthalate is selected from the group consisting of dimethyl phthalate.

10. The composition of claim 1, wherein the solvent comprises butyl carbitol.

11. The composition of claim 1, wherein the first inhibitor compound is present in the composition at a concentration of about 0% by weight to about 50% by weight, and the second inhibitor compound is present in the composition at a concentration of about 0% by weight to about 20% by weight.

12. The composition of claim 1, wherein the composition does not include a phenol, a nitroxyl radical, a nitroso group compound, or hydroquinone.

13. The composition of claim 1, further comprising a monomer selected from the group consisting of (meth)acrylic acid, methyl methacrylate, acrylic acid, acrylic acid esters, methacrylamide sulfate, vinyl acetate, acrylonitrile, acrolein, acrylates, methacrylates, 1,3-butadiene, styrene, isoprene, and combinations thereof.

14. A method of inhibiting polymerization of a monomer, the method comprising:
adding the composition of claim 1 to the monomer.

15. The method of claim 14, wherein the monomer is provided within a solution.

16. The method of claim 14, wherein the monomer is an ethylenically unsaturated monomer.

17. The method of claim 14, wherein the composition is added to the monomer such that a concentration of the first inhibitor compound and the second inhibitor compound in combination is about 0.1 ppm to about 10,000 ppm.

18. A composition comprising an inhibitor compound that is a phenothiazine derivative of formula (II): 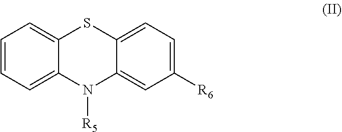
wherein R₅ is selected from the group consisting of hydrogen, alkyl, amine, aryl, and heteroaryl,
and wherein R₆ is selected from the group consisting of H, Cl, CF₃, and COCH₃,
and a solvent selected from the group consisting of a pyrrolidone, an ester that is a phthalate, a C₃-C₁₀, preferably, a C₅-C₁₀, ether such as butyl carbitol.

19. The composition of claim 18, wherein the solvent is N-methyl-2-pyrrolidone.

20. A method of inhibiting polymerization of a monomer, the method comprising:
adding the composition of claim 18 to the monomer.
